(19) United States
(12) Patent Application Publication
(10) Pub. No.: US 2010/0105702 A1
(43) Pub. Date: Apr. 29, 2010

(54) Heterocyclylethylamide derivatives

(76) Inventors: Darren Mansfield, Bergisch Gladbach (DE); Pierre-Yves Coqueron, Lyon (FR); Heiko Rieck, Burscheid (DE); Philippe Desbordes, Lyon (FR); Alain Villier, Collonges au Mont d'Or (FR); Marie-Claire Grosjean-Cournoyer, Curis au Mont d'Or (FR); Pierre Genix, Lyon (FR)

Correspondence Address: OSTROLENK FABER GERB & SOFFEN, 1180 AVENUE OF THE AMERICAS, NEW YORK, NY, 100368403, US

(21) Appl. No.: 11/919,624

(22) PCT Filed: May 2, 2006
(86) PCT No.: PCT/EP2006/061954

§ 371 Date: Feb. 15, 2008

(30) Foreign Application Priority Data
May 3, 2005 (EP) 05356076.9

(51) Int. Cl. A01N 43/54; C07D 403/12; A01P 3/00

(52) U.S. Cl. 514/256; 544/333

(57) ABSTRACT

A compound of general formula (I). A process for preparing this compound. A fungicide composition comprising a compound of general formula (I). A method for treating plants by applying a compound of general formula (I) or a composition comprising it.

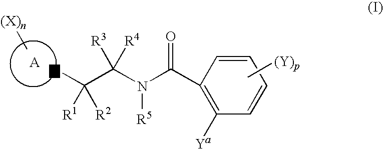

The present invention relates to novel heterocyclylethylamide derivatives, their process of preparation, their use as fungicides, particularly in the form of fungicidal compositions, and methods for the control of phytopathogenic fungi of plants using these compounds or their compositions.

International patent application WO2004/074280 discloses a broad family 2-pyridylethylcarbpxamide derivatives and their use as fungicides. This document does not disclose neither it covers heterocyclylethylbenzamide derivatives according to the present invention.

It is always of high-interest in the field of agrochemicals to use pesticidal compounds more active than the compounds already known by the man ordinary skilled in the art whereby less compound can be used whilst retaining equivalent efficacy.

Furthermore, the provision of new pesticidal compounds with a higher efficacy strongly reduces the risk of appearance of resistant strains in the fungi to be treated.

We have now found a new family of compounds which possess the above mentioned characteristics.

Accordingly, the present invention relates to a heterocyclylethylamide derivative of general formula (I)

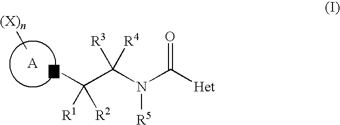

in which:
n is 1, 2, 3, 4 or 5;
A represents a 6-membered non-fused heterocycle with one, two or three heteroatoms which may be the same or different, provided that A is different from pyridyl, piperidine and piperazine;
z is a carbon atom or a heteroatom which can not be substituted by X;
X is the same or different and is a halogen atom, a nitro group, a cyano group, a hydroxy group, an amino group, a sulfanyl group, a pentafluoro-λ⁶-sulfanyl group, a formyl group, a formyloxy group, a formylamino group, a carboxy group, a carbamoyl group, a N-hydroxycarbamoyl group, a carbamate group, a (hydroxyimino)-C₁-C₆-alkyl group, a C₁-C₈-alkyl, a C₂-C₈-alkenyl, a C₂-C₈-alkynyl, a C₁-C₈-alkylamino, a C₁-C₈-halogenoalkyl having 1 to 5 halogen atoms, a di-C₁-C₈-alkylamino, a C₁-C₈-alkoxy, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₆-alkoxyimino, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a (benzyloxyimino)-C₁-C₆-alkyl, a benzyloxy, a benzylsulfanyl, a benzylamino, a phenoxy, a phenylsulfanyl a phenyl or a phenylamino;
R¹ and R² are the same or different and are a hydrogen atom a cyano group, a formyl group, a carboxy group, a carbamoyl group, a (hydroxyimino)-C₁-C₆-alkyl group, a C₁-C₈-alkyl, a C₃-C₇-cycloalkyl-C₁-C₈-alkyl, a C₂-C₈-alkenyl, a C₂-C₈-alkynyl, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-halogenoalkyl having 1 to 5 halogen atoms, a C₁-C₈-alkylcarbamoyl, a di-C₁-C₈-alkylcarbamoyl, a C₁-C₈-alkoxycarbamoyl, a C₁-C₈-alkoxycarbonyl or a C₁-C₈-halogenoalkoxycarbonyl having 1 to 5 halogen atoms;
R³ and R⁴ are the same or different and are a hydrogen atom, are a hydrogen atom, a hydrogen atom a cyano group, a formyl group, a carboxy group, a carbamoyl group, a (hydroxyimino)-C₁-C₆-alkyl group, a C₁-C₈-alkyl, C₃-C₇-cycloalkyl-C₁-C₈-alkyl, a C₂-C₈-alkenyl, a C₂-C₈-alkynyl, a C₃-C₈-cycloalkyl, a C₃-C₈-halogenocycloalkyl having 1 to 5 halogen atoms, a C₁-C₈-alkylcarbonyl, a C₁-C₈ halogenoalkylcarbonyl having 1 to 5 halogen atoms, a C₁-C₈-halogenoalkyl having 1 to 5 halogen atoms, a C₁-C₈-alkylcarbamoyl, a di-C₁-C₈-alkylcarbamoyl, a C₁-C₈-alkoxycarbamoyl, a C₁-C₈-alkoxycarbonyl or a C₁-C₈-halogenoalkoxycarbonyl having 1 to 5 halogen atoms;
R⁵ is a hydrogen atom, a C₁-C₆-alkyl, or a C₃-C₇-cycloalkyl;
Het represents 5-, 6- or 7-membered heterocycle with one, two or three heteroatoms which may be the same or different; Het being linked by a carbon atom and being at least substituted in ortho position;

as well as its salts, N-oxydes, metallic complexes, metalloidic complexes and optically active isomers.

In the context of the present invention:

halogen means fluorine, bromine, chlorine or iodine.

carboxy means —C(═O)OH; carbonyl means —C(═O)—; carbamoyl means —C(═O)NH₂; N-hydroxycarbamoyl means —C(═O)NHOH;

an alkyl group, an alkenyl group, and an alkynyl group as well as moieties containing these terms, can be linear or branched; and

heteroatom means sulphur, nitrogen or oxygen.

In the context of the present invention, it has also to be understood that in the case of di-substituted amino and of di-substituted carbamoyl radicals, the two substituents may form together with the nitrogen atom bearing them a saturated heterocyclic ring containing 3 to 7 atoms.

Any of the compounds of the present invention can exist in one or more optical or chiral isomer forms depending on the number of asymmetric centres in the compound. The invention thus relates equally to all the optical isomers and to their racemic or scalemic mixtures (the term “scalemic” denotes a mixture of enantiomers in different proportions), and to the mixtures of all the possible stereoisomers, in all proportions. The diastereoisomers and/or the optical isomers can be separated according to the methods which are known per se by the man ordinary skilled in the art.

Any of the compounds of the present invention can also exist in one or more geometric isomer forms depending on the number of double bonds in the compound. The invention thus relates equally to all geometric isomers and to all possible mixtures, in all proportions. The geometric isomers can be separated according to general methods, which are known per se by the man ordinary skilled in the art.

Any of the compounds of general formula (I) wherein X represents a hydroxy, a sulfanyl group or an amino group may be found in its tautomeric form resulting from the shift of the proton of said hydroxy, sulfanyl or amino group. Such tautomeric forms of such compounds are also part of the present invention. More generally speaking, all tautomeric forms of compounds of general formula (I) wherein X represents a hydroxy, a sulfanyl group or an amino group, as well as the tautomeric forms of the compounds which can optionally be used as intermediates in the preparation processes, and which will be defined in the description of these processes, are also part of the present invention.

According to the present invention, the “A” group may be substituted in any position by (X)ₙ, in which X and n are as defined above. Preferably, the present invention relates to heterocyclylethylamide derivative of general formula (I) in which the different characteristics may be chosen alone or in combination as being:

as regards n, n is 1 or 2;

as regards X, X is chosen as being a methyl group, a halogen atom or C₁-C₈-halogenoalkyl having 1 to 5 halogen atoms. More preferably X is a halogen atom.

According to the present invention, the carbon atoms of the carboxamide moiety of the compound of formula (I) are substituted by R¹, R², R³ and R⁴; R¹, R², R³ and R⁴ being as defined above. Preferably, the present invention also relates to heterocyclylethylamide derivative of general formula (I) in which the different characteristics may be chosen alone or in combination as being:

as regards R¹ and R², R¹ and R² are chosen, independently of each other, as being a hydrogen atom, a C₁-C₈-alkyl, a C₂-C₈-alkenyl, a C₂-C₈-alkynyl a C₃-C₈-cycloalkyl, a C₃-C₇-cycloalkyl-C₁-C₈-alkyl, a C₃-C₈-halogenocycloalkyl having 1 to 5 halogen atoms or a C₁-C₈-halogenoalkyl having 1 to 5 halogen atoms;

as regards R³ and R⁴, R³ and R⁴ are chosen, independently of each other, as being a a hydrogen atom, a C₁-C₈-alkyl, a C₂-C₈-alkenyl, a C₂-C₈-alkynyl, a C₃-C₈-cycloalkyl, a C₃-C₇-cycloalkyl-C₁-C₈-alkyl, a C₃-C₈-halogenocycloalkyl having 1 to 5 halogen atom or a C₁-C₈-halogenoalkyl having 1 to 5 halogen atoms.

According to the present invention, the nitrogen atom of the carboxamide moiety of the compound of formula (I) is substituted by R⁵, R⁵ being a hydrogen atom, a C₁-C₆-alkyl or a C₃-C₇-cycloalkyl. Preferably, the C₃-C₇-cycloalkyl is cyclopropyl.

According to the present invention, “Het” of the compound of general formula (I) is a 5-, 6- or 7-membered non-fused heterocycle with one, two or three heteroatoms which may be the same or different, Het being linked by a carbon atom and being substituted at least in ortho-position. Preferably, the present invention also relates to heterocyclylethylamide derivative of general formula (I) in which Het is chosen as being 2-furan, 3-furan, 4,5-dihydro-3-furan, 2-thiophene, 3-thiophene, 2-pyrrole, 3-pyrrole, 5-oxazole, 4-oxazole, 5-thiazole, 4-thiazole, 5-pyrazole, 4-pyrazole, 3-pyrazole, 3-isoxazole, 4-isoxazole, 5-isoxazole, 3-isothiazole, 4-1,2,3-triazole, 4-thiadiazole, 5-thiadiazole, 2-pyridine, 3-pyridine, 4-pyridine, 2-oxathiine, 4,5-dihydro-3-pyran, 4,5-dihydro-2-thiopyran, 4,5-dihydro-3-thiopyran or 2-pyrazine.

According to the present invention, “Het” of the compound of general formula (I) may be a five membered ring heterocycle. Specific examples of compounds of the present invention where Het is a five membered heterocycle include:

Het represents a heterocycle of the general formula (Het-1)

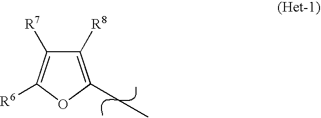

in which:
R⁶ and R⁷ may be the same or different and may be a hydrogen atom, a halogen atom, an amino group, a nitro group, a C₁-C₄-alkyl or a C₁-C₄-halogenoalkyl having 1 to 5 halogen atoms; and
R⁸ may be a halogen atom, a nitro group, a C₁-C₄-alkyl or a C₁-C₄-halogenoalkyl having 1 to 5 halogen atoms.

Het represents a heterocycle of the general formula (Het-2)

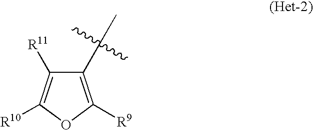

in which:
R⁹ may be a hydrogen atom, a halogen atom, a C₁-C₄-alkyl or a C₁-C₄-halogenoalkyl having 1 to 5 halogen atoms; and
R¹⁰ and R¹¹ may be the same or different and may be a hydrogen atom, a halogen atom, an amino group, a C₁-C₄-alkyl or a C₁-C₄-halogenoalkyl having 1 to 5 halogen atoms;

provided that the R⁹ and R¹¹ are not both a hydrogen atom.

Het represents a heterocycle of the general formula (Het-3)

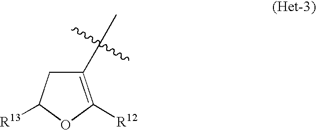

in which:
R¹² may be a halogen atom, a C₁-C₄-alkyl or a C₁-C₄-halogenoalkyl having 1 to 5 halogen atoms; and
R¹³ may be a hydrogen atom, a C₁-C₄-alkyl or a C₁-C₄-halogenoalkyl having 1 to 5 halogen atoms.

Het represents a heterocycle of the general formula (Het-4)

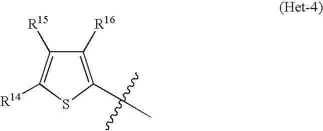

in which:
R¹⁴ and R¹⁵ may be the same or different and may be a hydrogen atom, a halogen atom, a C₁-C₄-alkyl, a C₁-C₄-halogenoalkyl having 1 to 5 halogen atoms, a C₁-C₄-alkylthio, a C₁-C₄-alkylsulphonyl, a phenyl optionally substituted by a halogen atom or a C₁-C₄-alkyl or a pyridyl optionally substituted by a halogen atom or a C₁-C₄-alkyl; and
R¹⁶ may be a halogen atom, a cyano group, a C₁-C₄-alkyl, a C₁-C₄-halogenoalkyl having 1 to 5 halogen atoms or a C₁-C₄-halogenoalkoxy having 1 to 5 halogen atoms.

Het represents a heterocycle of the general formula (Het-5)

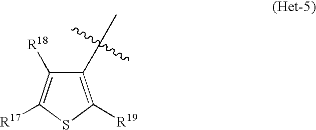

in which:
R¹⁷ and R¹⁸ may be the same or different and may be a hydrogen atom, a halogen atom, a C₁-C₄-alkyl, a C₁-C₄-allyloxy or a C₁-C₄-halogenoalkyl having 1 to 5 halogen atoms; and
R¹⁹ may be a hydrogen atom, a halogen atom, a C₁-C₄-alkyl or a C₁-C₄-halogenoalkyl having 1 to 5 halogen atoms;

provided that the R¹⁸ and R¹⁹ are not both a hydrogen atom.

Het represents a heterocycle of the general formula (Het-6)

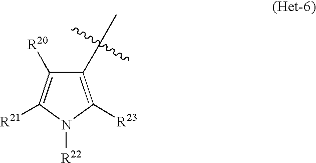

in which:
R²⁰ may be a hydrogen atom, a halogen atom, a cyano group, a C₁-C₄-alkyl or a C₁-C₄-halogenoalkyl having 1 to 5 halogen atoms;
R²¹ and R²³ may be the same or different and may be a hydrogen atom, a halogen atom, a C₁-C₄-alkyl or a C₁-C₄-halogenoalkyl having 1 to 5 halogen atoms; and
R²² may be a hydrogen atom, a cyano group, a C₁-C₄-alkyl, a C₁-C₄-halogenoalkyl having 1 to 5 halogen atoms, a C₁-C₄-alkoxy-C₁-C₄-alkyl, a hydroxy-C₁-C₄-alkyl, a C₁-C₄-alkylsulphonyl, a di(C₁-C₄-alkyl)aminosulphonyl, a C₁-C₆-alkylcarbonyl, a phenylsulphonyl optionally substituted by a halogen atom or a C₁-C₄-alkyl, or a benzoyl optionally substituted by a halogen atom or a C₁-C₄-alkyl;

provided that the R²⁰ and R²³ are not both a hydrogen atom.

Het represents a heterocycle of the general formula (Het-7)

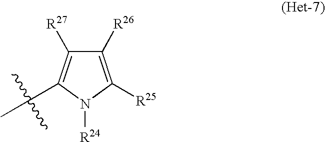

in which:
R²⁴ may be a hydrogen atom, a cyano group, a C₁-C₄-alkyl, a C₁-C₄-halogenoalkyl having 1 to 5 halogen atoms, a C₁-C₄-alkoxy-C₁-C₄-alkyl, a hydroxy-C₁-C₄-alkyl, a C₁-C₄-alkyl sulphonyl, a di(C₁-C₄-alkyl)aminosulphonyl, a C₁-C₆-alkylcarbonyl, a phenylsulphonyl optionally substituted by a halogen atom or a C₁-C₄-alkyl, or a benzoyl optionally substituted by a halogen atom or a C₁-C₄-alkyl; and
R²⁵, R²⁶ and R²⁷ may be the same or different and may be a hydrogen atom, a halogen atom, a cyano group, a C₁-C₄-alkyl, a C₁-C₄-halogenoalkyl having 1 to 5 halogen atoms or a C₁-C₄-alkylcarbonyl;

provided that R²⁴ and R²⁷ are not both a hydrogen atom.

Het represents a heterocycle of the general formula (Het-8)

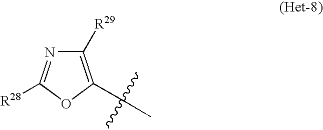

in which:
R²⁸ may be a hydrogen atom or a C₁-C₄-alkyl; and
R²⁹ may be a halogen atom, a C₁-C₄-alkyl or a C₁-C₄-halogenoalkyl having 1 to 5 halogen atoms.

Het represents a heterocycle of the general formula (Het-9)

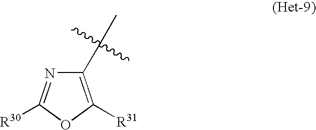

in which:
R³⁰ may be a hydrogen atom or a C₁-C₄-alkyl; and
R³¹ may be a halogen atom, a C₁-C₄-alkyl, a C₁-C₄-halogenoalkyl having 1 to 5 halogen atoms or a phenyl optionally substituted by a halogen atom or a C₁-C₄-alkyl.

Het represents a heterocycle of the general formula (Het-10)

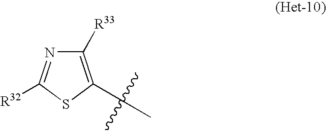

in which:
R³² may be a hydrogen atom, a halogen atom, an amino group, a cyano group, a C₁-C₄-alkylamino, a di-(C₁-C₄-alkylamino, a C₁-C₄-alkyl, a C₁-C₄-halogenoalkyl having 1 to 5 halogen atoms or a phenyl optionally substituted by a halogen atom or a C₁-C₄-alkyl; and
R³³ may be a halogen atom, a C₁-C₄-alkyl or a C₁-C₄-halogenoalkyl having 1 to 5 halogen atoms.

Het represents a heterocycle of the general formula (Het-11)

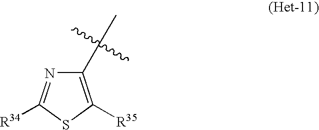

in which:
R³⁴ may be a hydrogen atom, a halogen atom, an amino group, a cyano group, a C₁-C₄-alkylamino, a di-(C₁-C₄-alkyl)amino, a C₁-C₄-alkyl or a C₁-C₄-halogenoalkyl having 1 to 5 halogen atoms; and
R³⁵ may be a halogen atom, a C₁-C₄-alkyl or a C₁-C₄-halogenoalkyl having 1 to 5 halogen atoms.

Het represents a heterocycle of the general formula (Het-12)

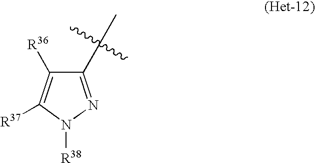

in which:
R³⁶ may be a halogen atom, a cyano group, a nitro group, a C₁-C₄-alkyl, a C₁-C₄-halogenoalkyl having 1 to 5 halogen atoms, a C₃-C₆-cycloalkyl, a C₁-C₄-alkoxy, a C₁-C₄-halogenoalkoxy having 1 to 5 halogen atoms, a C₁-C₄-alkylthio, a C₁-C₄-halogenoalkylthio having 1 to 5 halogen atoms, an aminocarbonyl group or an aminocarbonyl-C₁-C₄-alkyl;
R³⁷ may be a hydrogen atom, a halogen atom, a cyano group, a nitro group, a C₁-C₄-alkyl, a C₁-C₄-alkoxy or a C₁-C₄-alkylthio; and
R³⁸ may be a hydrogen atom, a phenyl, a C₁-C₄-alkyl, a C₁-C₄-halogenoalkyl having 1 to 5 halogen atoms, a hydroxy-C₁-C₄-alkyl, a C₂-C₆-alkenyl, a C₃-C₆-cycloalkyl, a C₁-C₄-alkylthio-C₁-C₄-alkyl, a C₁-C₄-halogenoalkylthio-C₁-C₄-alkyl having 1 to 5 halogen atoms, a C₁-C₄-alkoxy-C₁-C₄-alkyl or a C₁-C₄-halogenoalkoxy-C₁-C₄-alkyl having 1 to 5 halogen atoms.

Het represents a heterocycle of the general formula (Het-13)

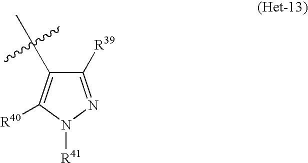

in which:
R³⁹ may be a hydrogen atom, a halogen atom, a cyano group, a nitro group, a C₁-C₄-alkyl, a C₁-C₄-halogenoalkyl having 1 to 5 halogen atoms, a C₃-C₆-cycloalkyl, a C₁-C₄-alkoxy, a C₁-C₄-halogenoalkoxy having 1 to 5 halogen atoms, a C₁-C₄-alkylthio, a C₁-C₄-halogenoalkylthio having 1 to 5 halogen atoms, an aminocarbonyl or an aminocarbonyl-C₁-C₄-alkyl;
R⁴⁰ may be a hydrogen atom, a halogen atom, a cyano group, a C₁-C₄-alkyl, a C₁-C₄-alkoxy, a C₁-C₄-halogenoalkoxy having 1 to 5 halogen atoms or a C₁-C₁-alkylthio; and
R⁴¹ may be a hydrogen atom, a C₁-C₄-alkyl, a C₁-C₄-halogenoalkyl having 1 to 5 halogen atoms, a hydroxy-C₁-C₄-alkyl, a C₂-C₆-alkenyl, a C₃-C₆-cycloalkyl, a C₁-C₄-alkylthio-C₁-C₄-alkyl, a C₁-C₄-halogenoalkylthio-C₁-C₄-alkyl having 1 to 5 halogen atoms, a C₁-C₄-alkoxy-C₁-C₄-alkyl, a C₁-C₄-halogenoalkoxy-C₁-C₄-alkyl having 1 to 5 halogen atoms or a phenyl optionally substituted by a halogen atom, a C₁-C₄-alkyl, a C₁-C₄-alkoxyalkyl or a nitro group;

provided that the R³⁹ and R⁴⁰ are not both a hydrogen atom.

Het represents a heterocycle of the general formula (Het-14)

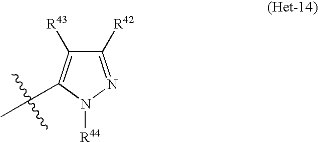

in which:
R⁴² may be a hydrogen atom, a halogen atom, a cyano group, a nitro group, a C₁-C₄-alkyl, a C₁-C₄-halogenoalkyl having 1 to 5 halogen atoms, a C₃-C₆-cycloalkyl, a C₁-C₄-alkoxy, a C₁-C₄-halogenoalkoxy having 1 to 5 halogen atoms, a C₁-C₄-alkylthio, a C₁-C₄-halogenoalkylthio having 1 to 5 halogen atoms, an aminocarbonyl, or an aminocarbonyl-C₁-C₄-alkyl;
R⁴³ may be a hydrogen atom, a halogen atom, a cyano group, a C₁-C₄-alkyl, a C₁-C₄-alkoxy, a C₁-C₄-alkylthio or a C₁-C₄-halogenoalkyl having 1 to 5 halogen atoms;
R⁴⁴ may be a hydrogen atom, a phenyl, a benzyl, a C₁-C₄-alkyl, a C₁-C₄-halogenoalkyl having 1 to 5 halogen atoms, a hydroxy-C₁-C₄-alkyl, a C₂-C₆-alkenyl, a C₃-C₆-cycloalkyl, a C₁-C₄-alkylthio-C₁-C₄-alkyl, a C₁-C₄-halogenoalkylthio-C₁-C₄-alkyl having 1 to 5 halogen atoms, a C₁-C₄-alkoxy-C₁-C₄-alkyl, a C₁-C₄-halogenoalkoxy-C₁-C₄-alkyl having 1 to 5 halogen atoms;

provided that R⁴³ and R⁴⁴ are not both a hydrogen atom.

Het represents a heterocycle of the general formula (Het-15)

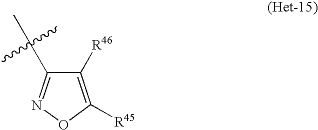

in which:
R⁴⁵ may be a hydrogen atom, a halogen atom, a C₁-C₄-alkyl or a C₁-C₄-halogenoalkyl having 1 to 5 halogen atoms; and
R⁴⁶ may be a halogen atom, a C₁-C₄-alkyl or a C₁-C₄-halogenoalkyl having 1 to 5 halogen atoms.

Het represents a heterocycle of the general formula (Het-16)

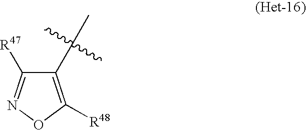

in which R⁴⁷ and R⁴⁸ may be the same or different and may be a hydrogen atom, a halogen atom, a C₁-C₄-alkyl, a C₁-C₄-halogenoalkyl having 1 to 5 halogen atoms, a phenyl optionally substituted by a halogen atom or a C₁-C₄-alkyl, or a heterocyclyl optionally substituted by a halogen atom or a C₁-C₄-alkyl;

provided that R⁴⁷ and R⁴⁸ are not both a hydrogen atom.

Het represents a heterocycle of the general formula (Het-17)

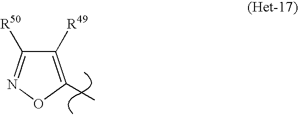

in which
R⁴⁹ may be a halogen atom, a C₁-C₄-alkyl or a C₁-C₄-halogenoalkyl having 1 to 5 halogen atoms. and
R⁵⁰ may be a halogen atom, a C₁-C₄-alkyl or a C₁-C₄-halogenoalkyl having 1 to 5 halogen atoms.

Het represents a heterocycle of the general formula (Het-18)

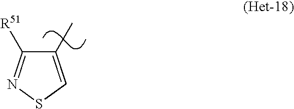

in which R⁵¹ may be a halogen atom, a C₁-C₄-alkyl or a C₁-C₄-halogenoalkyl having 1 to 5 halogen atoms.

Het represents a heterocycle of the general formula (Het-19)

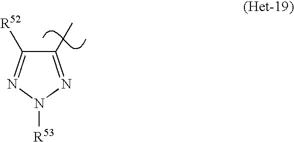

in which:
R⁵² may be a halogen atom, a C₁-C₄-alkyl or a C₁-C₄-halogenoalkyl having 1 to 5 halogen atoms; and
R⁵³ may be a hydrogen atom, a C₁-C₄-alkyl, a C₁-C₄-halogenoalkyl having 1 to 5 halogen atoms, or a phenyl optionally substituted by a halogen atom or a C₁-C₄-alkyl.

Het represents a heterocycle of the general formula (Het-20)

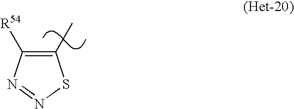

in which R⁵⁴ may be a halogen atom, a C₁-C₄-alkyl or a C₁-C₄-halogenoalkyl having 1 to 5 halogen atoms.

According to the present invention, “Het” of the compound of general formula (I) may be a six membered ring heterocycle. Specific examples of compounds of the present invention where Het is a six membered heterocycle include:

Het represents a heterocycle of the general formula (Het-21)

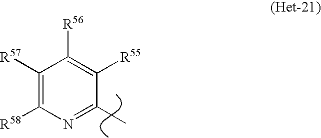

in which:
R⁵⁵ may be a halogen atom, a hydroxy group, a cyano group, a C₁-C₄-alkyl, a C₁-C₄-halogenoalkyl having 1 to 5 halogen atoms, a C₁-C₄-alkoxy, a C₁-C₄-alkylthio, a C₁-C₄-halogenoalkylthio having 1 to 5 halogen atoms or a C₁-C₄-halogenoalkoxy having 1 to 5 halogen atoms;
R⁵⁶, R⁵⁷ and R⁵⁸, which may be the same or different, may be a hydrogen atom, a halogen atom, a cyano group, a C₁-C₄-alkyl, a C₁-C₄-halogenoalkyl having 1 to 5 halogen atoms, a C₁-C₄-alkoxy, a C₁-C₄-alkylthio, a C₁-C₄-halogenoalkoxy having 1 to 5 halogen atoms, a C₁-C₄-alkylsulphinyl or a C₁-C₄-alkylsulphonyl.

Het represents a heterocycle of the general formula (Het-22)

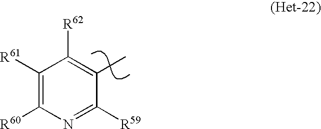

in which:
R⁵⁹ may be a hydrogen atom, a halogen atom, a hydroxy group, a cyano group, a C₁-C₄-alkyl, a C₁-C₄-halogenoalkyl having 1 to 5 halogen atoms, a C₁-C₄-alkoxy, a C₁-C₅-alkylthio, a C₂-C₅-alkenylthio a C₁-C₄-halogenoalkylthio having 1 to 5 halogen atoms, a C₁-C₄-halogenoalkoxy having 1 to 5 halogen atoms, a phenyloxy optionally substituted by a halogen atom or a C₁-C₄-alkyl, or a phenylthio optionally substituted by a halogen atom or a C₁-C₄-alkyl;
R⁶⁰, R⁶¹ and R⁶², which may the same or different, may be a hydrogen atom, a halogen atom, a cyano group, a C₁-C₄-alkyl, a C₁-C₄-halogenoalkyl having 1 to 5 halogen atoms, a C₁-C₄-alkoxy, a C₁-C₄-alkylthio, a C₁-C₄-halogenoalkoxy having 1 to 5 halogen atoms, a C₁-C₄-alkylsulphinyl, a C₁-C₄-alkylsulphonyl or a N-morpholine optionally substituted by a halogen atom or a C₁-C₄-alkyl, or a thienyl optionally substituted by a halogen atom or a C₁-C₄-alkyl;

provided that the R⁵⁹ and R⁶² are not both a hydrogen atom.

Het represents a heterocycle of the general formula (Het-23)

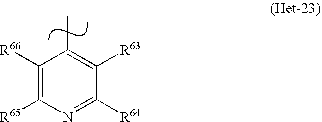

in which R⁶³, R⁶⁴, R⁶⁵ and R⁶⁶, which may be the same or different, may be a hydrogen atom, a halogen atom, a hydroxy group, a cyano group, a C₁-C₄-alkyl, a C₁-C₄-halogenoalkyl having 1 to 5 halogen atoms, a C₁-C₄-alkoxy, a C₁-C₄-alkylthio, a C₁-C₄-halogenoalkylthio having 1 to 5 halogen atoms, a C₁-C₄-halogenoalkoxy having 1 to 5 halogen atoms, a C₁-C₄-alkylsulphinyl or a C₁-C₄-alkylsulphonyl;

provided that the R⁶³ and R⁶⁶ are not both a hydrogen atom.

Het represents a heterocycle of the general formula (Het-24)

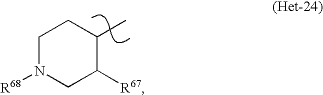

in which:
R⁶⁷ may be a halogen atom, a C₁-C₄-alkyl or a C₁-C₄-halogenoalkyl having 1 to 5 halogen atoms;
R⁶⁸ may be a hydrogen atom, a C₁-C₄-alkyl, a C₁-C₄-halogenoalkyl having 1 to 5 halogen atoms, a C₁-C₆-alkoxycarbonyl, a benzyl optionally substituted by 1 to 3 halogen atoms, a benzyloxycarbonyl optionally substituted by 1 to 3 halogen atoms or a heterocyclyl.

Het represents a heterocycle of the general formula (Het-25)

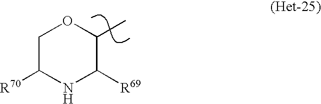

in which:
R⁶⁹ may be a halogen atom, a hydroxy group, a cyano group, a C₁-C₄-alkyl, a C₁-C₄-halogenoalkyl having 1 to 5 halogen atoms, a C₁-C₄-alkoxy, a C₁-C₄-alkylthio, a C₁-C₄-halogenoalkylthio having 1 to 5 halogen atoms or a C₁-C₄-halogenoalkoxy having 1 to 5 halogen atoms;
R⁷⁰ may be a hydrogen atom, a C₁-C₄-alkyl, a C₁-C₄-halogenoalkyl having 1 to 5 halogen atoms or a benzyl.

Het represents a heterocycle of the general formula (Het-26)

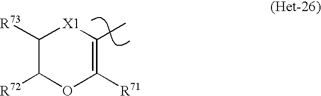

in which:
X¹ may be a sulphur atom, —SO—, —SO₂— or —CH₂—;
R⁷¹ may be a C₁-C₄-alkyl or a C₁-C₄-halogenoalkyl having 1 to 5 halogen atoms; and
R⁷² and R⁷³ may be the same or different and may be a hydrogen atom or a C₁-C₄-alkyl.

Het represents a heterocycle of the general formula (Het-27)

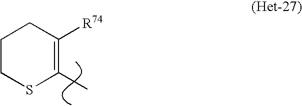

in which:
R⁷⁴ may be a C₁-C₄-alkyl or a C₁-C₄-halogenoalkyl having 1 to 5 halogen atoms;

Het represents a heterocycle of the general formula (Het-28)

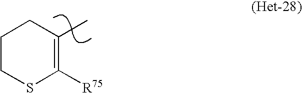

in which:
R⁷⁵ may be a C₁-C₄-alkyl or a C₁-C₄-halogenoalkyl having 1 to 5 halogen atoms.

Het represents a heterocycle of the general formula (Het-29)

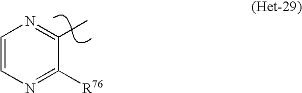

in which R⁷⁶ may be a halogen atom, a C₁-C₄-alkyl or a C₁-C₄-halogenoalkyl having 1 to 5 halogen atoms.

According to the present invention, the “A” group of the compound of general formula (I) is a six membered ring non-fused heterocycle with one, two or three heteroatoms which may be the same or different. Preferably, the present invention also relates to heterocyclylethylamide derivative of general formula (I) in which the different characteristics may be chosen alone or in combination as being:

A is chosen as being a morpholine, a pyrane, a dehydropyrane, a thiopyrane, a dehydrothiopyrane, a pyridazine, a pyrimidine or a pyrazine. More preferably, A is a pyrimidine. Even more preferably A is a 2-pyrimidine;

A is substituted in ortho position.

Specific examples of “A” group include:

A represents a heterocycle of the general formula (A-1)

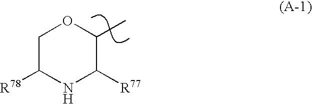

in which:
R⁷⁷ may be a halogen atom, a hydroxy group, a cyano group, a C₁-C₄-alkyl, a C₁-C₄-halogenoalkyl having 1 to 5 halogen atoms, a C₁-C₄-alkoxy, a C₁-C₄-alkylthio, a C₁-C₄-halogenoalkylthio having 1 to 5 halogen atoms or a C₁-C₄-halogenoalkoxy having 1 to 5 halogen atoms;
R⁷⁸ may be a hydrogen atom, a C₁-C₄-alkyl, a C₁-C₄-halogenoalkyl having 1 to 5 halogen atoms or a benzyl.

A represents a heterocycle of the general formula (A-2)

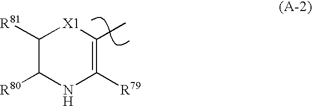

in which:
X¹ may be a sulphur atom, —SO—, —SO₂— or —CH₂—;
R⁷⁹ may be a C₁-C₄-alkyl or a C₁-C₄-halogenoalkyl having 1 to 5 halogen atoms; and
R⁸⁰ and R⁸¹ may be the same or different and may be a hydrogen atom or a C₁-C₄-alkyl.

A represents a heterocycle of the general formula (A-3)

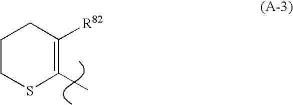

in which:
R⁸² may be a C₁-C₄-alkyl or a C₁-C₄-halogenoalkyl having 1 to 5 halogen atoms;

A represents a heterocycle of the general formula (A-4)

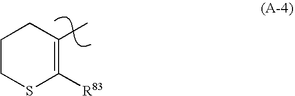

in which:
R⁸³ may be a C₁-C₄-alkyl or a C₁-C₄-halogenoalkyl having 1 to 5 halogen atoms.

A represents a heterocycle of the general formula (A-5)

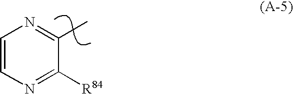

in which R⁸⁴ may be a halogen atom, a C₁-C₄-alkyl or a C₁-C₄-halogenoalkyl having 1 to 5 halogen atoms.

A represents a heterocycle of the general formula (A-6)

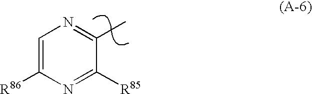

in which:
R⁸⁵ may be a hydrogen atom, a halogen atom, a C₁-C₄-alkyl or a C₁-C₄-halogenoalkyl having 1 to 5 halogen atoms; and
R⁸⁶ may be a halogen atom, a C₁-C₄-alkyl or a C₁-C₄-halogenoalkyl having 1 to 5 halogen atoms.

A represents a heterocycle of the general formula (A-7)

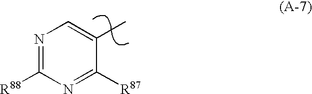

in which:
R⁸⁷ may be a hydrogen atom, a halogen atom, a C₁-C₄-alkyl or a C₁-C₄-halogenoalkyl having 1 to 5 halogen atoms; and
R⁸⁸ may be, a halogen atom, a C₁-C₄-alkyl or a C₁-C₄-halogenoalkyl having 1 to 5 halogen atoms.

A represents a heterocycle of the general formula (A-8)

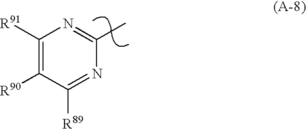

in which:
R⁸⁹ and R⁹¹ may be the same or different and may be a hydrogen atom, a halogen atom, a C₁-C₄-alkyl or a C₁-C₄-halogenoalkyl having 1 to 5 halogen atoms; and
R⁹⁰ may be a halogen atom, a C₁-C₄-alkyl or a C₁-C₄-halogenoalkyl having 1 to 5 halogen atoms.

A represents a heterocycle of the general formula (A-9)

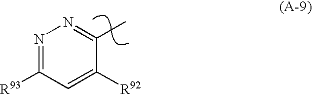

in which:
R⁹² may be a hydrogen atom, a halogen atom, a C₁-C₄-alkyl or a C₁-C₄-halogenoalkyl having 1 to 5 halogen atoms; and
R⁹³ may be a halogen atom, a C₁-C₄-alkyl or a C₁-C₄-halogenoalkyl having 1 to 5 halogen atoms.

A represents a heterocycle of the general formula (A-10)

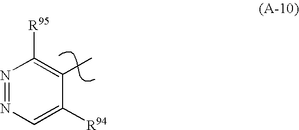

in which:
R⁹⁴ may be a hydrogen atom, a halogen atom, a C₁-C₄-alkyl or a C₁-C₄-halogenoalkyl having 1 to 5 halogen atoms; and
R⁹⁵ may be a halogen atom, a C₁-C₄-alkyl or a C₁-C₄-halogenoalkyl having 1 to 5 halogen atoms.

The present invention also relates to a process for the preparation of the compound of general formula (I). Thus, according to a further aspect of the present invention there is provided a process for the preparation of compound of general to formula (I) as defined above, which comprises reacting a heterocyclyl-ethylamine derivative of general formula (II) or one of its salt:

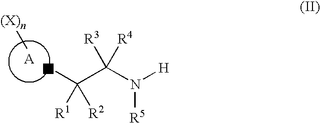

in which A, X, n, z, R¹, R², R³, R⁴ and R⁵ are as defined above; with a carboxylic acid derivative of the general formula (III)

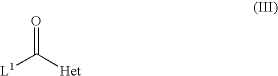

in which:
Het is as defined above; and
L¹ is a leaving group chosen as being a halogen atom, a hydroxyl group, —OR⁹⁶, —OCOR⁹⁶, R⁹⁶ being a C₁-C₆ alkyl, a C₁-C₆ haloalkyl, a benzyl, 4-methoxybenzyl, pentafluorophenyl or a group of formula

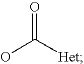

in the presence of a catalyst and, if L¹ is a hydroxyl group, in the presence of a condensing agent.

The process according to the present invention is conducted in the presence of a catalyst. Suitable catalyst may be chosen as being 4-dimethyl-aminopyridine, 1-hydroxy-benzotriazole or dimethylformamide.

In case L¹ is a hydroxy group, the process according to the present invention is conducted in the presence of condensing agent. Suitable condensing agent may be chosen as being acid halide former, such as phosgene, phosphorous tribromide, phosphorous trichloride, phosphorous pentachloride, phosphorous trichloride oxide or thionyl chloride; anhydride former, such as ethyl chloroformate, methyl chloroformate, isopropyl chloroformate, isobutyl chloroformate or methanesulfonyl-chloride; carbodiimides, such as N,N′-dicyclohexylcarbodiimide (DCC) or other customary condensing agents, such as phosphorous pentoxide, polyphosphoric acid, N,N′-carbonyl-diimidazole, 2-ethoxy-N-ethoxycarbonyl-1,2-dihydroquinoline (EEDQ), triphenylphosphine/tetrachloromethane, 4-(4,6-dimethoxy[1.3.5]triazin-2-yl)-4-methylmorpholinium chloride hydrate or bromo-tripyrrolidino-phosphonium-hexafluorophosphate.

When R⁵ is a hydrogen atom, the above mentioned process for the preparation of compound of general formula (I) may optionally be completed by a further step according to the following reaction scheme:

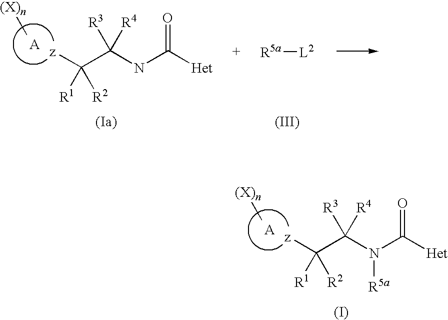

in which:
A, X, n, z, R¹, R², R³, R⁴ and Het are as defined above;
R^{5a} is a C₁-C₆-alkyl, or a C₃-C₇-cycloalkyl; and
L² is a leaving group chosen as being a halogen atom, a 4-methyl phenylsulfonyloxy or a methylsulfonyloxy;
   comprising the reaction of a compound of general formula (Ia) with a compound of general formula (III) to provide a compound of general formula (I).

Depending on the definition of R¹, R², R³, R⁴ or R⁵, amine derivatives of general formula (II) may be prepared by different processes. One example (A) of such a process may be when:
A, z, X, n are as defined above;
R¹ and R² are independently chosen as being a C₁-C₆ alkyl or a C₃-C₇ cycloalkyl-C₁-C₆ alkyl
R³, R⁴ and R⁵ are hydrogen atoms;
   then, the amine derivative of general formula (II) may be prepared according to a process which comprises:

a first step according to reaction scheme A-1:

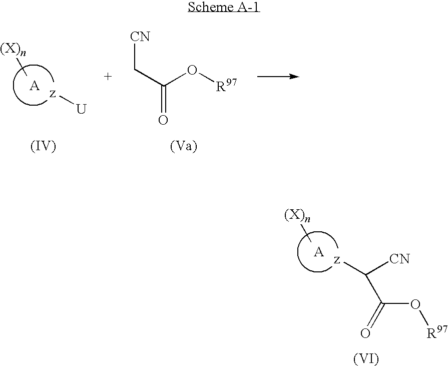

in which:
A, z, X and n are as defined above;
R⁹⁷ is a C₁-C₆ alkyl, a C₁-C₆ haloalkyl, a benzyl, 4-methoxybenzyl or pentafluorophenyl;
U is a leaving group chosen as being a halogen, a C₁-C₆ alkylsulfonate or a C₁-C₆ haloalkylsulfonate;
   comprising the arylation of a cyanoacetate derivative of general formula (Va) by a heterocylic derivative of general formula (IV), to provide a heterocyclyl-cyanoacetate derivative of general formula (VI), in the presence of a base, at a temperature of from 0° C. to 200° C.;

a second step according to reaction scheme A-2:

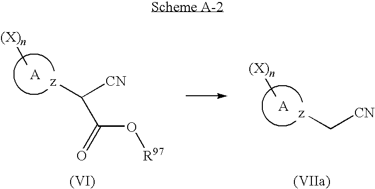

in which:
A, z, X and n are as defined above;
R⁹⁷ is a C₁-C₆ alkyl, a C₁-C₆ haloalkyl, a benzyl, 4-methoxybenzyl or pentafluorophenyl;
   comprising a basic hydrolysis, an acidic hydrolysis or a displacement by an halide of a compound of general formula (VI) in the same or a different pot to provide, upon heating at a temperature of from 40° C. to reflux, a heterocyclyl-acetonitrile derivative of general formula (VIIa);

a third step according to reaction scheme A-3:

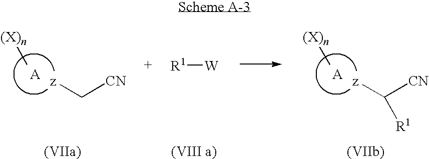

in which:
A, z, X and n are as defined above;
R¹ is a C₁-C₆ alkyl or a C₃-C₇ cycloalkyl-C₁-C₆ alkyl,
W is a halogen atom, a C₁-C₆ alkylsulfonate, a C₁-C₆ haloalkylsulfonate or a 4-methyl-phenylsulfonate;
   comprising the alkylation of a compound of general formula (VII a) by a reagent of general formula (VIII a) to provide a compound of general formula (VII b);

a fourth step according to reaction scheme A-4:

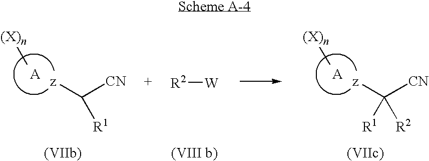

in which:
A, z, X and n are as defined above;
R¹, R² are a C₁-C₆ alkyl or a C₃-C₇ cycloalkyl-C₁-C₆ alkyl,
W is a halogen atom, a C₁-C₆ alkylsulfonate, a C₁-C₆ haloalkylsulfonate or a 4-methyl-phenylsulfonate;
   comprising the alkylation of a compound of general formula (VII b) by a reagent of general formula (VIII b) to provide a compound of general formula (VII c);

a fifth step according to reaction scheme A-5:

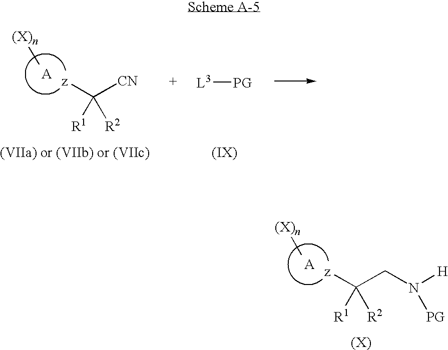

in which:
X, n are as defined above;
R¹, R² are a hydrogen atom, a C₁-C₆ alkyl or a C₃-C₇ cycloalkyl-C₁-C₆ alkyl,
L³ is a leaving group chosen as being a —OR⁹⁸ group or a —OCOR⁹⁸ group, R⁹⁸ being a C₁-C₆ alkyl, a C₁-C₆ haloalkyl, a benzyl, 4-methoxybenzyl or pentafluorophenyl;
PG represents a protecting group which may be a —COOR⁹⁹ group or —COR⁹⁹ group, R⁹⁹ being a C₁-C₆ alkyl, a C₁-C₆ haloalkyl, a benzyl, 4-methoxybenzyl or pentafluorophenyl;
   comprising the reduction, by hydrogenation or by an hydride donor, of a compound of general formula (VIIa), (VIIb) or (VIII), in the presence of a catalyst and in the presence of a compound of general formula (IX) to produce a compound of general formula (X), at a temperature of from 0° C. to 150° C. and under a pressure of from 1 bar and 100 bar;

a sixth step according to reaction scheme A-6:

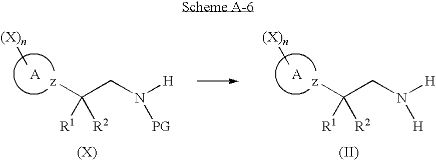

in which:
A, z, X and n are as defined above;
R¹, R² are a hydrogen atom, a C₁-C₆ alkyl or a C₃-C₇ cycloalkyl-C₁-C₆ alkyl,
PG represents a protecting group which may be a —COOR¹⁰⁰ group or —COR¹⁰⁰ group, R¹⁰⁰ being a C₁-C₆ alkyl, a C₁-C₆ haloalkyl, a benzyl, 4-methoxybenzyl or pentafluorophenyl;
   comprising a deprotection reaction, in an acidic or in a basic medium, of a compound of general formula (X) to provide an amine derivative of general formula (II) or one of its salt.

The first step (step A-1) is conducted in the presence of a base. Preferably, the base will be chosen as being an inorganic or an organic base. Suitable examples of such bases may for example be alkaline earth metal or alkali metal hydrides, hydroxides, amides, alcoholates, carbonates or hydrogen carbonates, acetates or tertiary amines.

The first step (step A-1) according to the present invention is conducted at a temperature of from 0° C. to 200° C. Preferably, first step (step A-1) is conducted at a temperature of from 0° C. to 120° C., more preferably at a temperature of from 0° C. to 80° C.

The first step (step A-1) according to the present invention may be conducted in the presence of a solvent. Preferably, the solvent is chosen as being water, an organic solvent or a mixture of both. Suitable organic solvents may for example be aliphatic, alicyclic or aromatic solvent.

The first step (step A-1) according to the present invention may also be conducted in the presence of a catalyst. Preferably, the catalyst is chosen as being palladium salts or complexes. More preferably, the catalyst is chosen as being a palladium complex. Suitable palladium complex catalyst may for example be generated directly in the reaction mixture by separately adding to the reaction mixture a palladium salt and a complex ligand. Suitable ligands may for example be bulky phosphines or arsines ligands, such as (R)-(−)-1-[(S)-2-(dicyclohexylphosphino)ferrocenyl]ethyldicyclohexylphosphine and its corresponding enantiomer, or a mixture of both; (R)-(−)-1-[(S)-2-(dicyclohexylphosphino)ferrocenyl]ethyldiphenylphosphine and its corresponding enantiomer, or a mixture of both; (R)-(−)-1 [(S)-2-(diphenylphosphino)ferrocenyl]ethyldi-t-butylphosphine and its corresponding enantiomer, or a mixture of both; or (R)-(−)-1-[(S)-2-(diphenylphosphino)ferrocenyl]ethyldicyclohexylphosphine and its corresponding enantiomer, or a mixture of both.

The fifth step (step A-5) according to the present invention is conducted in the presence of a hydride donor. Preferably, the hydride donor is chosen as being metal or metallloid hydrides such as LiAlH₄, NaBH₄, KBH₄, B₂H₆.

The fifth step (step A-5) according to the present invention is conducted in the presence of a catalyst. Preferably, the catalyst is chosen as being Co(II)-Chloride, Ni(II)-chloride, ammonia or one of its salt, Palladium on charcoal, Raney Nickel, Raney Cobalt or Platinum.

The fifth step (step A-5)) according to the present invention is conducted at a temperature of from 0° C. to 150° C. Preferably the temperature is of from 10° C. to 120° C. More preferably, the temperature is of from 10° C. to 80° C.

The fifth step (step A-5) according to the present invention is conducted under a pressure of from 1 bar to 100 bar. Preferably the pressure is of from 1 bar to 50 bar.

The fifth step (step A-5) according to the present invention may be conducted in the presence of an organic solvent, of water or of a mixture thereof. Preferably, the solvent is chosen as being ether, alcohol, carboxylic acid, or a mixture thereof with water or pure water.

The compound according to the present invention can be prepared according to the general processes of preparation described above. It will nevertheless be understood that, on the basis of his general knowledge and of available publications, the skilled worker will be able to adapt this method according to the specifics of each of the compounds, which it is desired to synthesise.

On the basis of his general knowledge and of available publications, the skilled worker will also be able to prepare intermediate compound of formula (V) according to the present invention.

The present invention also relates to a fungicidal composition comprising an effective amount of an active material of general formula (I). Thus, according to the present invention, there is provided a fungicidal composition comprising, as an active ingredient, an effective amount of a compound of general formula (I) as defined above and an agriculturally acceptable support, carrier or filler.

In the present specification, the term “support” denotes a natural or synthetic, organic or inorganic material with which the active material is combined to make it easier to apply, notably to the parts of the plant. This support is thus generally inert and should be agriculturally acceptable. The support may be a solid or a liquid. Examples of suitable supports include clays, natural or synthetic silicates, silica, resins, waxes, solid fertilisers, water, alcohols, in particular butanol, organic solvents, mineral and plant oils and derivatives thereof. Mixtures of such supports may also be used.

The composition may also comprise additional components. In particular, the composition may further comprise a surfactant. The surfactant can be an emulsifier, a dispersing agent or a wetting agent of ionic or non-ionic type or a mixture of such surfactants. Mention may be made, for example, of polyacrylic acid salts, lignosulphonic acid salts, phenolsulphonic or naphthalenesulphonic acid salts, polycondensates of ethylene oxide with fatty alcohols or with fatty acids or with fatty amines, substituted phenols (in particular alkylphenols or arylphenols), salts of sulphosuccinic acid esters, taurine derivatives (in particular alkyl taurates), phosphoric esters of polyoxyethylated alcohols or phenols, fatty acid esters of polyols, and derivatives of the above compounds containing sulphate, sulphonate and phosphate functions. The presence of at least one surfactant is generally essential when the active material and/or the inert support are water-insoluble and when the vector agent for the application is water. Preferably, surfactant content may be comprised between 5% and 40% by weight of the composition.

Optionally, additional components may also be included, e.g. protective to colloids, adhesives, thickeners, thixotropic agents, penetration agents, stabilisers, sequestering agents. More generally, the active materials can be combined with any solid or liquid additive, which complies with the usual formulation techniques.

In general, the composition according to the invention may contain from 0.05 to 99% (by weight) of active material, preferably 10 to 70% by weight.

Compositions according to the present invention can be used in various forms such as aerosol dispenser, capsule suspension, cold fogging concentrate, dustable powder, emulsifiable concentrate, emulsion oil in water, emulsion water in oil, encapsulated granule, fine granule, flowable concentrate for seed treatment, gas (under pressure), gas generating product, granule, hot fogging concentrate, macrogranule, microgranule, oil dispersible powder, oil miscible flowable concentrate, oil miscible liquid, paste, plant rodlet, powder for dry seed treatment, seed coated with a pesticide, soluble concentrate, soluble powder, solution for seed treatment, suspension concentrate (flowable concentrate), ultra low volume (ulv) liquid, ultra low volume (ulv) suspension, water dispersible granules or tablets, water dispersible powder for slurry treatment, water soluble granules or tablets, water soluble powder for seed treatment and wettable powder.

These compositions include not only compositions which are ready to be applied to the plant or seed to be treated by means of a suitable device, such as a spraying or dusting device, but also concentrated commercial compositions which must be diluted before application to the crop.

The compounds of the invention can also be mixed with one or more insecticides, fungicides, bactericides, attractant acaricides or pheromones or other compounds with biological activity. The mixtures thus obtained have a broadened spectrum of activity. The mixtures with other fungicides are particularly advantageous. Examples of suitable fungicide mixing partners may be selected in the following lists:

1) a compound capable to inhibit the nucleic acid synthesis like benalaxyl, benalaxyl-M, bupirimate, chiralaxyl, clozylacon, dimethirimol, ethirimol, furalaxyl, hymexazol, mefenoxam, metalaxyl, metalaxyl-M, ofurace, oxadixyl, oxolinic acid;

2) a compound capable to inhibit the mitosis and cell division like benomyl, carbendazim, diethofencarb, ethaboxam, fuberidazole, pencycuron, thiabendazole thiophanate-methyl, zoxamide;

3) a compound capable to inhibit the respiration for example

as CI-respiration inhibitor like diflumetorim;

as CII-respiration inhibitor like boscalid, carboxin, fenfuram, flutolanil, furametpyr, furmecyclox, mepronil, oxycarboxine, penthiopyrad, thifluzamide;

as CII-respiration inhibitor like amisulbrom, azoxystrobin, cyazofamid, dimoxystrobin, enestrobin, famoxadone, fenamidone, fluoxastrobin, kresoxim-methyl, metominostrobin, orysastrobin, picoxystrobin, pyraclostrobin, trifloxystrobin;

4) a compound capable of to act as an uncoupler like dinocap, fluazinam, meptyldinocap;

5) a compound capable to inhibit ATP production like fentin acetate, fentin chloride, fentin hydroxide, silthiofam;

6) a compound capable to inhibit AA and protein biosynthesis like andoprim, blasticidin-S, cyprodinil, kasugamycin, kasugamycin hydrochloride hydrate, mepanipyrim, pyrimethanil;

7) a compound capable to inhibit the signal transduction like fenpiclonil, fludioxonil, quinoxyfen;

8) a compound capable to inhibit lipid and membrane synthesis like biphenyl, chlozolinate, edifenphos, iodocarb, iprobenfos, iprodione, isoprothiolane, procymidone, propamocarb, propamocarb hydrochloride, pyrazophos, tolclofos-methyl, vinclozolin;

9) a compound capable to inhibit ergosterol biosynthesis like aldimorph, azaconazole, bitertanol, bromuconazole, cyproconazole, diclobutrazole, difenoconazole, diniconazole, diniconazole-M, dodemorph, dodemorph acetate, epoxiconazole, etaconazole, fenarimol, fenbuconazole, fenhexamid, fenpropidin, fenpropimorph, fluquinconazole, flurprimidol, flusilazole, flutriafol, furconazole, furconazole-cis, hexaconazole, imazalil, imazalil sulfate, imibenconazole, ipconazole, metconazole, myclobutanil, naftifine, nuarimol, oxpoconazole, paclobutrazol, pefurazoate, penconazole, prochloraz, propiconazole, prothioconazole, pyributicarb, pyrifenox, simeconazole, spiroxamine, tebuconazole, terbinafine, tetraconazole, triadimefon, triadimenol, tridemorph, triflumizole, triforine, triticonazole, uniconazole, viniconazole, voriconazole;

10) a compound capable to inhibit cell wall synthesis like benthiavalicarb, bialaphos, dimethomorph, flumorph, iprovalicarb, polyoxins, polyoxorim, validamycin A;

11) a compound capable to inhibit melanine biosynthesis like carpropamid, diclocymet, fenoxanil, phthalide, pyroquilon, tricyclazole;

12) a compound capable to induce a host defence like acibenzolar-S-methyl, probenazole, tiadinil;

13) a compound capable to have a multisite action like Bordeaux mixture, captafol, captan, chlorothalonil, copper naphthenate, copper oxide, copper oxychloride, copper preparations such as copper hydroxide, copper sulphate, dichlofluanid, dithianon, dodine, dodine free base, ferbam, fluorofolpet, folpet, guazatine, guazatine acetate, iminoctadine, iminoctadine albesilate, iminoctadine triacetate, mancopper, mancozeb, maneb, metiram, metiram zinc, oxine-copper, propineb, sulphur and sulphur preparations including calcium polysulphide, thiram, tolylfluanid, zineb, ziram;

14) a compound selected in the following list: (2E)-2-(2-{[6-(3-chloro-2-methylphenoxy)-5-fluoropyrimidin-4-yl]oxy}phenyl)-2-(methoxyimino)-N-methylacetamide, (2E)-2-{2-[({[(1E)-1-(3-{[(E)-1-fluoro-2-phenylvinyl]oxy}phenyl)ethylidene]amino}oxy)methyl]phenyl}-2-(methoxyimino)-N-methylacetamide, 1-(4-chlorophenyl)-2-(1H-1,2,4-triazol-1-yl)cycloheptanol, 1-[(4-methoxyphenoxy)methyl]-2,2-dimethylpropyl 1H-imidazole-1-carboxylate, 2-(4-chlorophenyl)-N-{2-[3-methoxy-4-(prop-2-yn-1-yloxy)phenyl]ethyl}-2-(prop-2-yn-1-yloxy)acetamide, 2,3,5,6-tetrachloro-4-(methylsulfonyl)pyridine, 2-butoxy-6-iodo-3-propyl-4H-chromen-4-one, 2-chloro-N-(1,1,3-trimethyl-2,3-dihydro-1H-inden-4-yl)nicotinamide, 2-phenylphenol and salts, 3,4,5-trichloropyridine-2,6-dicarbonitrile, 3,4-dichloro-N-(2-cyanophenyl)isothiazole-5-carboxamide, 3-[5-(4-chlorophenyl)-2,3-dimethylisoxazolidin-3-yl]pyridine, 5-chloro-6-(2,4,6-trifluorophenyl)-N-[(1R)-1,2,2-trimethylpropyl][1,2,4]triazolo[1,5-a]pyrimidin-7-amine, 5-chloro-7-(4-methylpiperidin-1-yl)-6-(2,4,6-trifluorophenyl)[1,2,4]triazolo[1,5-a]pyrimidine, 5-chloro-N-[(1R)-1,2-dimethylpropyl]-6-(2,4,6-trifluorophenyl) [1,2,4]triazol o[1,5-a]pyrimidin-7-amine, 8-hydroxyquinoline sulfate, benthiazole, bethoxazin, capsimycin, carvone, chinomethionat, cufraneb, cyflufenamid, cymoxanil, dazomet, debacarb, dichlorophen, diclomezine, dicloran, difenzoquat, difenzoquat methylsulphate, diphenylamine, ferimzone, flumetover, fluopicolide, fluoroimide, flusulfamide, fosetyl-aluminium, fosetyl-calcium, fosetyl-sodium, hexachlorobenzene, inunamycin, methasulfocarb, methyl (2-chloro-5-{(1E)-N-[(6-methylpyridin-2-yl)methoxy]ethanimidoyl}benzyl)carbamate, methyl (2E)-2-{2[({cyclopropyl[(4-methoxyphenyl)imino]methyl}thio)methyl]phenyl}-3-methoxyacrylate, methyl 1-(2,2-dimethyl-2,3-dihydro-1H-inden-1-yl)-1H-imidazole-5-carboxylate, methyl 3-(4-chlorophenyl)-3-{[N-(isopropoxycarbonyl)valyl]amino}propanoate, methyl isothiocyanate, metrafenone, mildiomycin, N-(3′, 4′-dichloro-5-fluorobiphenyl-2-yl)-3-(difluoromethyl)-1-methyl-1H-pyrazole-4-carboxamide, N-(3-ethyl-3,5,5-trimethylcyclohexyl)-3-(formylamino)-2-hydroxybenzamide, N-(4-chloro-2-nitrophenyl)-N-ethyl-4-methylbenzenesulfonamide, N-[(5-bromo-3-chloropyridin-2-yl)methyl]-2,4-dichloronicatinamide, N-[1-(5-bromo-3-chloropyridin-2-yl)ethyl]-2,4-dichloronicotinamide, N-[1-(5-bromo-3-chloropyridin-2-yl)ethyl]-2-fluoro-4-iodonicotinamide, N-[2-(4-{[3-(4-chlorophenyl)prop-2-yn-1-yl]oxy}-3-methoxyphenyl)ethyl]-N<sup>2</sup>-(methylsulfonyl)valinamide, N-{(Z)-[(cyclopropylmethoxy)imino][6-(difluoromethoxy)-2,3-difluorophenyl]methyl}-2-phenylacetamide, N-{2-[3-chloro-5-(trifluoromethyl)pyridin-2-yl]ethyl}-2-(trifluoromethyl)benzamide, natamycin, nickel dimethyldithiocarbamate, nitrothal-isopropyl, O-{1-[(4-methoxyphenoxy)methyl]-2,2-dimethylpropyl} 1H-imidazole-1-carbothioate, octhilinone, oxamocarb, oxyfenthiin, pentachlorophenol and salts, phosphorous acid and its salts, piperalin, propamocarb fosetylate, propanosine-sodium, proquinazid, pyrrolnitrine, quintozene, tecloftalam, tecnazene, triazoxide, trichlamide and zarilamid.

The composition according to the invention comprising a mixture of a compound of formula (I) with a bactericide compound may also be particularly advantageous. Examples of suitable bactericide mixing partners may be selected in the following list: bronopol, dichlorophen, nitrapyrin, nickel dimethyldithiocarbamate, kasugamycin, octhilinone, furancarboxylic acid, oxytetracycline, probenazole, streptomycin, tecloftalam, copper sulphate and other copper preparations.

The fungicidal compositions of the present invention can be used to curatively or preventively control the phytopathogenic fungi of crops. Thus, according to a further aspect of the present invention, there is provided a method for curatively or preventively controlling the phytopathogenic fungi of crops characterised in that a fungicidal composition as hereinbefore defined is applied to the seed, the plant and/or to the fruit of the plant or to the soil in which the plant is growing or in which it is desired to grow.

The composition as used against phytopathogenic fungi of crops comprises an effective and non-phytotoxic amount of an active material of general formula (I).

The expression “effective and non-phytotoxic amount” means an amount of composition according to the invention which is sufficient to control or destroy the fungi present or liable to appear on the crops, and which does not entail any appreciable symptom of phytotoxicity for the said crops. Such an amount can vary within a wide range depending on the fungus to be controlled, the type of crop, the climatic conditions and the compounds included in the fungicidal composition according to the invention.

This amount can be determined by systematic field trials, which are within the capabilities of a person skilled in the art.

The method of treatment according to the present invention is useful to treat propagation material such as tubers or rhizomes, but also seeds, seedlings or seedlings pricking out and plants or plants pricking out. This method of treatment can also be useful to treat roots. The method of treatment according to the present invention can also be useful to treat the overground parts of the plant such as trunks, stems or stalks, leaves, flowers and fruits of the concerned plant.

Among the plants that can be protected by the method according to the present invention, mention may be made of cotton; flax; vine; fruit or vegetable crops such as Rosaceae sp. (for instance pip fruit such as apples and pears, but also stone fruit such as apricots, almonds and peaches), Ribesioidae sp., Juglandaceae sp., Betulaceae sp., Anacardiaceae sp., Fagaceae sp., Moraceae sp., Oleaceae sp., Actimidaceae sp., Lauraceae sp., Musaceae sp. (for instance banana trees and plantins), Rubiaceae sp., Theaceae sp., Sterculiceae sp., Rutaceae sp. (for instance lemons, oranges and grapefruit); Solanaceae sp. (for instance tomatoes), Liliaceae sp., Asteraceae sp. (for instance lettuces), Umbelliferae sp., Cruciferae sp., Chenopodiaceae sp., Cucurbitaceae sp., Papilionaceae sp. (for instance peas), Rosaceae sp. (for instance strawberries); major crops such as Graminae sp. (for instance maize, lawn or cereals such as wheat, rice, barley and triticale), Asteraceae sp. (for instance sunflower), Cruciferae sp. (for instance colza), Fabacae sp. (for instance peanuts), Papilionaceae sp. (for instance soybean), Solanaceae sp. (for instance potatoes), Chenopodiaceae sp. (for instance beetroots); horticultural and forest crops; as well as genetically modified homologues of these crops.

Among the diseases of plants or crops that can be controlled by the method according to the present invention, mention may be made of:

Powdery mildew diseases such as:

Blumeria diseases, caused for example by Blumeria graminis;

Podosphaera diseases, caused for example by Podosphaera leucotricha;

Sphaerotheca diseases, caused for example by Sphaerotheca fuliginea;

Uncinula diseases, caused for example by Uncinula necator;

Rust diseases such as:

Gymnosporangium diseases, caused for example by Gymnosporangium sabinae;

Hemileia diseases, caused for example by Hemileia vastatrix;

Phakopsora diseases, caused for example by Phakopsora pachyrhizi or Phakopsora meibomiae;

Puccinia diseases, caused for example by Puccinia recondita;

Uromyces diseases, caused for example by Uromyces appendiculatus;

Oomycete diseases such as:

Bremia diseases, caused for example by Bremia lactucae;

Peronospora diseases, caused for example by Peronospora pisi or P. brassicae;

Phytophthora diseases, caused for example by Phytophthora infestans;

Plasmopara diseases, caused for example by Plasmopara viticola;

Pseudoperonospora diseases, caused for example by Pseudoperonospora humuli or

Pseudoperonospora cubensis;

Pythium diseases, caused for example by Pythium ultimum;

Leafspot, leaf blotch and leaf blight diseases such as:

Alternaria diseases, caused for example by Alternaria solani;

Cercospora diseases, caused for example by Cercospora beticola;

Cladiosporum diseases, caused for example by Cladiosporium cucumerinum;

Cochliobolus diseases, caused for example by Cochliobolus sativus;

Colletotrichum diseases, caused for example by Colletotrichum lindemuthanium;

Cycloconium diseases, caused for example by Cycloconium oleaginum;

Diaporthe diseases, caused for example by Diaporthe citri;

Elsinoe diseases, caused for example by Elsinoe fawcettii;

Gloeosporium diseases, caused for example by Gloeosporium laeticolor,

Glomerella diseases, caused for example by Glomerella cingulata;

Guignardia diseases, caused for example by Guignardia bidwelli;

Leptosphaeria diseases, caused for example by Leptosphaeria maculans; Leptosphaeria nodorum;

Magnaporthe diseases, caused for example by Magnaporthe grisea;

Mycosphaerella diseases, caused for example by Mycosphaerella graminicola; Mycosphaerella arachidicola; Mycosphaerella fijiensis;

Phaeosphaeriai diseases, caused for example by Phaeosphaeria nodorum;

Pyrenophora diseases, caused for example by Pyrenophora teres;

Ramularia diseases, caused for example by Ramularia collo-cygni;

Rhynchosporium diseases, caused for example by Rhynchosporium secalis;

Septoria diseases, caused for example by Septoria apii or Septoria lycopercisi;

Typhula diseases, caused for example by Typhula incarnata;

Venturia diseases, caused for example by Venturia inaequalis;

Root and stem diseases such as:

Corticium diseases, caused for example by Corticium graminearum;

Fusarium diseases, caused for example by Fusarium oxysporum;

Gaeumannomyces diseases, caused for example by Gaeumannomyces graminis;

Rhizoctonia diseases, caused for example by Rhizoctonia solani;

Tapesia diseases, caused for example by Tapesia acuformis;

Thielaviopsis diseases, caused for example by Thielaviopsis basicola;

Ear and panicle diseases such as:

Alternaria diseases, caused for example by Alternaria spp.;

Aspergillus diseases, caused for example by Aspergillus flavus;

Cladosporium diseases, caused for example by Cladosporium spp.;

Claviceps diseases, caused for example by Claviceps purpurea;

Fusarium diseases, caused for example by Fusarium culmorum;

Gibberella diseases, caused for example by Gibberella zeae;

Monographella diseases, caused for example by Monographella nivalis;

Smut and bunt diseases such as:

Sphacelotheca diseases, caused for example by Sphacelotheca reiliana;

Tilletia diseases, caused for example by Tilletia caries;

Urocystis diseases, caused for example by Urocystis occulta;

Ustilago diseases, caused for example by Ustilago nuda;

Fruit rot and mould diseases such as:

Aspergillus diseases, caused for example by Aspergillus flavus;

Botrytis diseases, caused for example by Botrytis cinerea;

Penicillium diseases, caused for example by Penicillium expansum;

Sclerotinia diseases, caused for example by Sclerotinia sclerotiorum;

Verticilium diseases, caused for example by Verticilium alboatrum;

Seed and soilbome decay, mould, wilt, rot and damping-off diseases:

Fusarium diseases, caused for example by Fusarium culmorum;

Phytophthora diseases, caused for example by Phytophthora cactorumi;

Pythium diseases, caused for example by Pythium ultimum;

Rhizoctonia diseases, caused for example by Rhizoctonia solani;

Sclerotium diseases, caused for example by Sclerotium rolfsii;

Microdochium diseases, caused for example by Microdochium nivale;

Canker, broom and dieback diseases such as:

Nectria diseases, caused for example by Nectria galligena;

Blight diseases such as:

Monilinia diseases, caused for example by Monilinia laxa;

Leaf blister or leaf curl diseases such as:

Taphrina diseases, caused for example by Taphrina deformans;

Decline diseases of wooden plants such as:

Esca diseases, caused for example by Phaemoniella clamydospora;

Diseases of flowers and Seeds such as:

Botrytis diseases, caused for example by Botrytis cinerea;

Diseases of tubers such as:

Rhizoctonia diseases, caused for example by Rhizoctonia solani.

The fungicide composition according to the present invention may also be used against fungal diseases liable to grow on or inside timber. The term “timber” means all types of species of wood, and all types of working of this wood intended for construction, for example solid wood, high-density wood, laminated wood, and plywood. The method for treating timber according to the invention mainly consists in contacting one or more compounds of the present invention, or a composition according to the invention; this includes for example direct application, spraying, dipping, injection or any other suitable means.

The dose of active material usually applied in the treatment according to the present invention is generally and advantageously between 10 and 800 g/ha, preferably between 50 and 300 g/ha for applications in foliar treatment. The dose of active substance applied is generally and advantageously between 2 and 200 g per 100 kg of seed, preferably between 3 and 150 g per 100 kg of seed in the case of seed treatment. It is clearly understood that the doses indicated above are given as illustrative examples of the invention. A person skilled in the art will know how to adapt the application doses according to the nature of the crop to be treated.

The fungicidal composition according to the present invention may also be used in the treatment of genetically modified organisms with the compounds according to the invention or the agrochemical compositions according to the invention. Genetically modified plants are plants into whose genome a heterologous gene encoding a protein of interest has been stably integrated. The expression “heterologous gene encoding a protein of interest” essentially means genes which give the transformed plant new agronomic properties, or genes for improving the agronomic quality of the transformed plant.

The compositions according to the present invention may also be used for the preparation of composition useful to curatively or preventively treat human and animal fungal diseases such as, for example, mycoses, dermatoses, trichophyton diseases and candidiases or diseases caused by Aspergillus spp., for example Aspergillus fumigatus.

The aspects of the present invention will now be illustrated with reference to the following tables of compounds and examples. The following Table illustrates in a non-limiting manner examples of fungicidal compounds according to the present invention. In the following Examples, M+1 (or M−1) means the molecular ion peak, plus or minus 1 a.m.u. (atomic mass units) respectively, as observed in mass spectroscopy and M (ApcI+) means the molecular ion peak as it was found via positive atmospheric pressure chemical ionisation in mass spectroscopy.

TABLE A

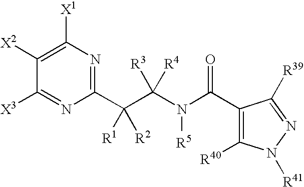

| Compound X | ¹X | ²X | ³R | ¹R | ²R | ³R | ⁴R | ⁵R | ³⁹R | ⁴⁰R | ⁴¹ | M + 1 |
|---|---|---|---|---|---|---|---|---|---|---|---|---|
| A-1 | H | Br | H | H | H | H | H | H | CHF₂ | H | Me | 362 |

### EXAMPLES OF PROCESS FOR THE PREPARATION OF THE COMPOUND OF GENERAL FORMULA (I)

### Synthesis of N-[2-(5-bromo-1,2-dihydropyrimidin-2-yl)ethyl]-3-difluoromethyl)-1-methyl-1,1-pyrazole-4-carboxamide (Compound A-1)

### Synthesis of ethyl (5-bromopyrimidin-2-yl)(cyano)acetate

To a chilled stirred suspension of sodium hydride (60% in oil, 34.55 mmol) in tetrahydrofuran (20 mL) was added 1.17 g of ethyl cyanoacetate (10.34 mmol) to portionwise over 10 minutes. After effervescence has ceased, the yellow cloudy mixture was left to warm up to room temperature whilst stirring.2.00 g of 5-bromo,2-bromo-pyrimidine (10.34 mmol) in 30 mL of tetrahydrofuran was added. And the mixture was left to stir at room temperature for six hours. The resultant brown mixture was concentrated under reduced pressure to give a bright brown precipitate to which water (200 mL) was added. After extraction with heptane (200 mL), the aqueous phase was acidified by the addition of HCl 1M and the resultant precipitate was collected by filtration to give on drying ethyl (5-bromopyrimidin-2-yl)(cyano)acetate as a bright brown powder (0.67 g, yield=24%).

¹H NMR δ (ppm, DMSO-d₆): 3.5 (b, 1H); 8.9 (b, 1H); 8.45 (b, 1H); 4.24 (t, 2H); 1.25 (t, 3H).

### Synthesis of (5-bromopyrimidin-2-yl)acetonitrile

To 2.70 g of ethyl (5-bromopyrimidin-2-yl)(cyano)acetate (9.970 mmol) in dimethylsulfoxyde (20 ml) was added 0.584 g of sodium chloride (9.970 mmol) and 0.18 mL of water (9.970 mmol). The mixture was heated at 120° C. under a nitrogen atmosphere for 3 hours. The mixture was poured into water (300 mL) and the resultant precipitate was collected by filtration to give 5-bromopyrimidin-2-yl)acetonitrile (0.6 g, yield=30%).

The clear yellow filtrate was salted by the addition of a saturated solution of sodium chloride, neutralided with sodium hydroxide, to give a red solution which was then extracted with dichloromethane. The organic phase was collected, dried over magnesium sulphate, and evaporated in vacuo to give a dark oil, which was purified by flash chromatography using 2/1 heptane/ethyl acetate as eluent, yielding to 5-bromopyrimidin-2-yl)acetonitrile (0.9 g, yield=43%).

¹H NMR δ (ppm, CDCl₃): 9.0 (s, 2H); 4.25 (s, 2H).

### Synthesis of tert-butyl [2-(5-bromopyrimidin-2-yl)ethyl]carbamate

To a cold (−5-0° C.), stirred mixture of the 5-bromopyrimidin-2-yl)acetonitrile (0.9 g), tert-butyloxy-carbonate (1.98 g; 2 equivs), and nickel chloride hexahydrate (1.08 g) under a nitrogen atmosphere was added, portionwise, sodium borohydride (0.86 g; 5 equivs). After the addition the mixture was left to stir, whilst warming to room temperature, for 6 hrs and left to stand overnight.

The mixture was diluted with ethyl acetate (200 mls) and filtered through kieselghur. The filtrate was washed with brine and the organic phase was collected, dried over MgSO₄, and evaporated in vacuo to give the product as a brown oil.

The crude product was purified by flash chromatography to yield to 0.22 g of tert-butyl [2-(5-bromopyrimidin-2-yl)ethyl]carbamate (16%)

¹H NMR δ (ppm, CDCl₃): 8.76 (s, 2H); 5.22 (b, NH); 3.64 (m, 2H); 3.13 (m, 2H); 1.43 (s, 9H).

### Synthesis of N-[2-(5-bromo-1,2-dihydropyrimidin-2-yl)ethyl]-3-difluoromethyl)-1-methyl-1H-pyrazole-4-carboxamide (Compound A-1)

To a clear pale yellow mixture of the tert-butyl [2-(5-bromopyrimidin-2-yl)ethyl]carbamate (0.22 g, 0.7 mmol) in dichloromethane (2 ml) was added trifluoroacetic acid (10 equivs) and the mixture was left to stir at room temperature for 7 hrs and left to stand overnight. Saturated sodium bicarbonate solution was added until the effervescence stopped (to give a basic medium). The organic phase was collected, dried over MgSO4, and evaporated in vacuo after filtration. to yield to 0.12 g of 2-(5-bromopyrimidin-2-yl)ethanamine (80%) which is used directly without purification.

To 0.06 g of 2-(5-bromopyrimidin-2-yl)ethanamine (3.0 mmol) and triethylamine (0.03 g, 1.0 equiv.) in THF (5 ml), at room temperature whilst stirring, was added the 3-(difluoromethyl)-1-methyl-1H-pyrazole-4-carbonyl chloride (0.058 g, 1.0 equiv.) and the mixture was left to stir at room temperature overnight. The solvent was removed by evaporation in vacuo and saturated sodium bicarbonate (10 mls) was added. The mixture was extracted with dichloromethane and the organic phase was collected, dried over MgSO₄, and evaporated in vacuo to give the crude product which was purified by chromatography on silica using 1:1 heptane/ethyl acetate to yield to 0.028 g of N-[2-(5-bromo-1,2-dihydropyrimidin-2-yl)ethyl]-3-difluoromethyl)-1-methyl-1H-pyrazole-4-carboxamide (Compound A-1) (26%) Mass Spectrum: [M+3]=362.

1. A compound of general formula (I) 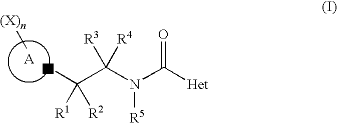
in which:
n is 1, 2, 3, 4 or 5;
A represents a 6-membered non-fused heterocycle with one, two or three heteroatoms which may be the same or different, provided that A is different from pyridyl, piperidine and piperazine;
z is a carbon atom or a heteroatom which can not be substituted by X;
X is the same or different and is a halogen atom, a nitro group, a cyano group, a hydroxy group, an amino group, a sulfanyl group, a pentafluoro-λ⁶-sulfanyl group, a formyl group, a formyloxy group, a formylamino group, a carboxy group, a carbamoyl group, a N-hydroxycarbamoyl group, a carbamate group, a (hydroxyimino)-C₁-C₆-alkyl group, a C₁-C₈-alkyl, a C₂-C₈-alkenyl, a C₂-C₈-alkynyl, a C₁-C₈-alkylamino, a C₁-C₈-halogenoalkyl having 1 to 5 halogen atoms, a di-C₁-C₈-alkylamino, a C₁-C₈-alkoxy, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-allylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₆-alkoxyimino, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a (benzyloxyimino)-C₁-C₆-allyl, a benzyloxy, a benzylsulfanyl, a benzylamino, a phenoxy, a phenylsulfanyl a phenyl or a phenylamino;
R¹ and R² are the same or different and are a hydrogen atom a cyano group, a formyl group, a carboxy group, a carbamoyl group, a (hydroxyimino)-C₁-C₆-alkyl group, a C₁-C₈-alkyl, a C₃-C₇-cycloalkyl-C₁-C₈-alkyl, a C₂-C₈-alkenyl, a C₂-C₈-alkynyl, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C_{g}-halogenoalkyl having 1 to 5 halogen atoms, a C₁-C₈-alkylcarbamoyl, a di-C₁-C₈-allylcarbamoyl, a C₁-C₈-alkoxycarbamoyl, a C₁-C₈-alkoxycarbonyl or a C₁-C₈-halogenoalkoxycarbonyl having 1 to 5 halogen atoms;
R³ and R⁴ are the same or different and are a hydrogen atom, are a hydrogen atom, a hydrogen atom a cyano group, a formyl group, a carboxy group, a carbamoyl group, a (hydroxyimino)-C₁-C₆-alkyl group, a C₁-C₈-alkyl, C₃-C₇-cycloalkyl-C₁-C₈-alkyl, a C₂-C₈-alkenyl, a C₂-C₈-alkynyl, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-halogenoalkyl having 1 to 5 halogen atoms, a C₁-C₈-allylcarbamoyl, a di-C₁-C₈-allylcarbamoyl, a C₁-C₈-alkoxycarbamoyl, a C₁-C₈-alkoxycarbonyl or a C₁-C₈-halogenoalkoxycarbonyl having 1 to 5 halogen atoms;
R⁵ is a hydrogen atom, a C₁-C₆-alkyl, or a C₃-C₇-cycloalkyl;
Het represents 5-, 6- or 7-membered heterocycle with one, two or three heteroatoms which may be the same or different; Het being linked by a carbon atom and being at least substituted in ortho position;
as well as its salts, N-oxydes, metallic complexes, metalloidic complexes and optically active isomers.

2. A compound according to claim 1, characterised in that n is 1 or 2.

3. A compound according to claim 1 or 2, characterised in that X is chosen as being a hydrogen atom, a methyl group or a halogen atom, a C₁-C₈-halogenoalkyl having 1 to 5 halogen atoms.

4. A compound according to any of the claims 1 to 3, characterised in that R¹ and R² are chosen, independently of each other, as being a hydrogen atom, a C₁-C₈-alkyl, a C₂-C₈-alkenyl, a C₂-C₈-alkynyl, a C₃-C₈-cycloalkyl, a C₃-C₇-cycloalkyl-C₁-C₈-alkyl, a C₃-C₈-halogenocycloalkyl having 1 to 5 halogen atoms or a C₁-C₈-halogenoalkyl having 1 to 5 halogen atoms;

5. A compound according to any of the claims 1 to 4, characterised in that R³ and R⁴ are chosen, independently of each other, as being a hydrogen atom, a C₁-C₈-alkyl, a C₂-C₈-alkenyl, a C₂-C₈-alkynyl, a C₃-C₈-cycloalkyl, a C₃-C₇-cycloalkyl-C₁-C₈-alkyl, a C₃-C₈-halogenocycloalkyl having 1 to 5 halogen atom or a C₁-C₈-halogenoalkyl having 1 to 5 halogen atoms.

6. A compound according to any of the claims 1 to 5, characterised in that Het is a six membered ring heterocycle.

7. A compound according to any of the claims 1 to 5, characterised in that Het is a five membered ring heterocycle.

8. A compound according to any of the claims 1 to 5, characterised in that Het is chosen as being 2-furan, 3-furan, 4,5-dihydro-3-furan, 2-thiophene, 3-thiophene, 2-pyrrole, 3-pyrrole, 5-oxazole, 4-oxazole, 5-thiazole, 4-thiazole, 5-pyrazole, 4-pyrazole, 3-pyrazole, 3-isoxazole, 4-isoxazole, 5-isoxazole, 3-isothiazole, 4-1,2,3-triazole, 4-thiadiazole, 5-thiadiazole, 2-pyridine, 3-pyridine, 4-pyridine, 2-oxathiine, 4,5dihydro-3-pyran, 4,5-dihydro-2-thiopyran, 4,5dihydro-3-thiopyran or 2-pyrazine.

9. A compound according to any of the claims 1 to 8, characterised in that A is chosen as being a morpholine, a pyrane, a dehydropyrane, a thiopyrane, a dehydrothiopyrane, a pyridazine, a pyrimidine or a pyrazine.

10. A compound according to any of the claims 1 to 9, characterised in that A is substituted in ortho position.

11. A process for the preparation of a compound of general formula (I) as defined in any of the claims 1 to 10, which comprises reacting a heterocyclylethylamine derivative of general formula (II) or one of its salt: 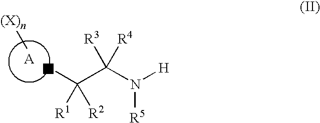
in which A, X, n, z, R¹, R², R³, R⁴ and R⁵ are as defined in claim 1;
with a carboxylic acid derivative of the general formula (III) 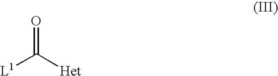
in which:
Het is as defined in claim 1; and
L¹ is a leaving group chosen as being a halogen atom, a hydroxyl group, —OR⁹⁶, —OCOR⁹⁶, R⁹⁶ being a C₁-C₆ alkyl, a C₁-C₆ haloalkyl, a benzyl, 4-methoxybenzyl, pentafluorophenyl or a group of formula 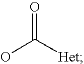
in the presence of a catalyst and, if L¹ is a hydroxyl group, in the presence of a condensing agent.

12. A process according to claim 11, characterised in that R⁵ is a hydrogen atom, and that the process is completed by a further step according to the following reaction scheme: 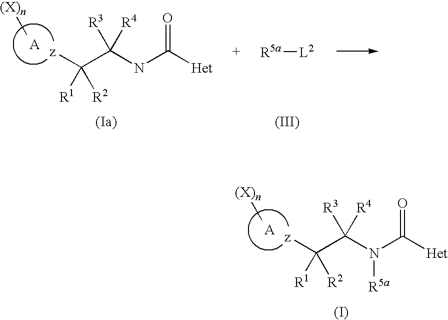
in which:
A, X, n, z, R¹, R², R³, R⁴ and Het are as defined in claim 1;
R^{5a} is a C₁-C₆-alkyl, or a C₃-C₇-cycloalkyl; and
L² is a leaving group chosen as being a halogen atom, a 4-methyl phenylsulfonyloxy or a methylsulfonyloxy;
comprising the reaction of a compound of general formula (Ia) with a compound of general formula (III) to provide a compound of general formula (I).

13. Fungicide composition comprising an effective amount of a compound according to any of the claims 1 to 10 and an agriculturally acceptable support.

14. Method for preventively or curatively combating the phytopathogenic fungi of crops, characterised in that an effective and non-phytotoxic amount of a composition according to claim 13 is applied to the plant seeds or to the plant leaves and/or to the fruits of the plants or to the soil in which the plants are growing or in which it is desired to grow them.
